(19) United States
(12) Patent Application Publication
(10) Pub. No.: US 2023/0330193 A1
(43) Pub. Date: Oct. 19, 2023

(54) ENHANCEMENT OF STEM CELL THERAPY FOR CARTILAGE DEGENERATION BY ANTI-OXIDANT PRE-CONDITIONING

(71) Applicant: CREATIVE MEDICAL TECHNOLOGIES, INC., Phoenix, AZ (US)

(72) Inventors: Thomas ICHIM, San Diego, CA (US); Amit PATEL, Salt Lake City, UT (US); Courtney BARTLETT, Phoenix, AZ (US)

(73) Assignee: CREATIVE MEDICAL TECHNOLOGIES, INC., Phoenix, AZ (US)

(21) Appl. No.: 18/300,222

(22) Filed: Apr. 13, 2023

Related U.S. Application Data
(60) Provisional application No. 63/331,183, filed on Apr. 14, 2022.

(51) Int. Cl. A61K 38/44; A61K 33/04; A61K 38/06; A61K 33/00; A61K 31/436; A61K 35/28; A61K 45/06

(52) U.S. Cl. CPC A61K 38/446; C12Y 115/01001; A61K 33/04; A61K 38/063; A61K 33/00; A61K 31/436; A61K 35/28; A61K 45/06

(57) ABSTRACT

Disclosed are means of augmenting efficacy of stem cell therapy for articular tissues through introduction of a step to reduce oxidative stress inside the joint before administration of stem cells. In one embodiment reduction of oxidative stress and/or inflammation is accomplished by administration of anti-oxidant agents locally in the tissue in which stem cell administration is anticipated to. In one embodiment administration of hydrogen gas is provided as a “preconditioning” step before administration of stem cell therapy. In one embodiment cell therapy is a same day non manipulated procedure involving administration of bone marrow aspirate.

### CROSS-REFERENCE TO RELATED APPLICATIONS

This application claims priority to U.S. Provisional Application Ser. No. 63/331,183, titled “Enhancement of Stem Cell Therapy for Cartilage Degeneration by Anti-Oxidant Pre-Conditioning”, filed Apr. 14, 2022, which is hereby incorporated by reference in its entirety.

### FIELD OF THE INVENTION

The teachings herein are directed to methods of enchancing stem cells in order to administer to patients suffering from cartilage degeneration.

### BACKGROUND

The body is known to possess different regenerative compartments within tissues. The most commonly known one is bone marrow which produces approximately 5 billion blood cells per minute. Within the bone marrow stem cells reside in hypoxic niches. When stem cells are taken out of hypoxic areas and oxygen tension is increased, there is a correlative decrease in regenerative potential. In the area of other regenerative tissues, there has been little studies to determine the effects of oxygen tension on regenerative activity.

Current conventional research dealing to osteoarthritis (OA) has been focused on the studies identifying the mechanism of cartilage degeneration which is the cause of the arthritis. Accordingly, the main factors leading to the degeneration mechanism are well known. Existing treatment strategies also focus on slowing the progression of the disease by suppressing degeneration factors, and these strategies cannot have the fundamental therapeutic effect on regenerating cartilage. Cartilage tissue is a tissue that gradually degrades when it begins to be damaged by aging or injury. An estimated 10%-15% of all adults over the age of 60 will develop some degree of OA (over 30 million adults in the US), with prevalence being higher among women, and on the rise due to the ageing of the populations and obesity. OA is also a source of morbidity and economic loss in the racehorse and companion animal populations. Aside from osteoarthritis, a large population of younger subjects are afflicted with injuries to cartilage or ligaments of the joints. Common acute injuries of the joint involve damage to anterior and posterior cruciate ligament (ACL and PCL), medial and lateral collateral ligaments (MCL and LCL), and menisci.

Unfortunately, drugs or other therapies used to treat degenerative arthritis remain at pain relief levels such as hyaluronic acid and anti-inflammatory drugs. The treatments that induce fundamental regeneration of cartilage have not yet been developed, and research is in its infancy.

### SUMMARY

Preferred embodiments are directed to methods of enhancing stem cell activity in the treatment of cartilage degenerative conditions though administering at least one or more antioxidants and/or anti-inflammatory agents before, at the same time has, and subsequent to stem cell therapy.

Preferred methods include embodiments wherein said cartilage degenerative condition is osteoarthritis.

Preferred methods include embodiments wherein said cartilage degenerative condition is immunologically mediated.

Preferred methods include embodiments wherein said cartilage degenerative condition is mediated by injury.

Preferred methods include embodiments wherein said cartilage degenerative condition is mediated by genetic predisposition.

Preferred methods include embodiments wherein said antioxidant is super oxide dismutase.

Preferred methods include embodiments wherein said super oxide dismutase is manganese dependent.

Preferred methods include embodiments wherein the gene encoding manganese dependent superoxide dismutase is administered intra-articularly.

Preferred methods include embodiments wherein said gene encoding manganese dependent superoxide dismutase is transfected into articular tissue by means of a viral vector.

Preferred methods include embodiments wherein said gene encoding manganese dependent superoxide dismutase is transfected into articular tissue by means of a hydrodynamic transfection.

Preferred methods include embodiments wherein said gene encoding manganese dependent superoxide dismutase is transfected into articular tissue by means of a mRNA liposomal delivery.

Preferred methods include embodiments wherein said gene encoding manganese dependent superoxide dismutase is transfected into articular tissue by means of naked DNA administration.

Preferred methods include embodiments wherein said antioxidant is zinc.

Preferred methods include embodiments wherein said antioxidant is vitamin C.

Preferred methods include embodiments wherein said antioxidant is vitamin E.

Preferred methods include embodiments wherein said antioxidant is selenium.

Preferred methods include embodiments wherein said antioxidant is Trolox.

Preferred methods include embodiments wherein said antioxidant is ebselen.

Preferred methods include embodiments wherein said antioxidant is glutathione.

Preferred methods include embodiments wherein said antioxidant is carotene.

Preferred methods include embodiments wherein said antioxidant is ubiquinol.

Preferred methods include embodiments wherein said antioxidant is propyl gallate.

Preferred methods include embodiments wherein said antioxidant is hydrogen gas.

Preferred methods include embodiments wherein said antioxidant is xenon gas.

Preferred methods include embodiments wherein said antioxidant is argon gas.

Preferred methods include embodiments wherein said antioxidant is neon gas.

Preferred methods include embodiments wherein said antioxidant is krypton gas.

Preferred methods include embodiments wherein said antioxidant is butylated hydroxytoluene.

Preferred methods include embodiments wherein said antioxidant is butylated hydroxyanisole.

Preferred methods include embodiments wherein said antioxidant is butylated hydrogen sulfide.

Preferred methods include embodiments wherein said antioxidant is erythrobate.

Preferred methods include embodiments wherein said antioxidant is sodium tripolyphosphate.

Preferred methods include embodiments wherein said antioxidant is ethylenediaminetetraacetic acid.

Preferred methods include embodiments wherein said antioxidant is ethoxyquin.

Preferred methods include embodiments wherein said antioxidant is casein.

Preferred methods include embodiments wherein said antioxidant is pyruvate.

Preferred methods include embodiments wherein said antioxidant is minocycline.

Preferred methods include embodiments wherein said antioxidant is tetracyclin.

Preferred methods include embodiments wherein said anti-inflammatory agent is hydroxychloroquine.

Preferred methods include embodiments wherein said anti-inflammatory agent is an NF-kappa B inhibitor.

Preferred methods include embodiments wherein said NF-kappa B inhibitor is hydroxychloroquine.

Preferred methods include embodiments wherein said NF-kappa B inhibitor is Calagualine.

Preferred methods include embodiments wherein said NF-kappa B inhibitor is Conophylline.

Preferred methods include embodiments wherein said NF-kappa B inhibitor is Evodiamine.

Preferred methods include embodiments wherein said NF-kappa B inhibitor is Geldanamycin.

Preferred methods include embodiments wherein said NF-kappa B inhibitor is selected from a group comprising of: Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, Saline (low Na+ istonic).

Preferred methods include embodiments wherein an inhibitor of inflammatory cytokines is administered intra-articularly prior to, concurrent with, or subsequent to stem cell administration.

Preferred methods include embodiments wherein said inflammatory cytokines are selected from a group comprised of: inflammatory cytokines are cytokines capable of inducing expression of genes in endothelial cells selected from a group comprising of: IL-6, Myosin 1, IL-33, Hypoxia Inducible Factor-1, Guanylate Binding Protein Isoform I, Aminolevulinate delta synthase 2, AMP deaminase, IL-17, DNAJ-like 2 protein, Cathepsin L, Transcription factor-20, M31724, pyenylalkylamine binding protein; HEC, GA17, arylsulfatase D gene, arylaulfatase E gene, cyclin protein gene, pro-platelet basic protein gene, PDGFRA, human STS WI-12000, mannosidase, beta A, lysosomal MANBA gene, UBE2D3 gene, Human DNA for Ig gamma heavy-chain, STRL22, BHMT, homo sapiens Down syndrome critical region, FI5613 containing ZNF gene family member, IL8, ELFR, homo sapiens mRNA for dual specificity phosphatase MKP-5, homo sapiens regulator of G protein signaling 10 mRNA complete, Homo sapiens Wnt-13 Mma, homo sapiens N-terminal acetyltransferase complex ardl subunit, ribosomal protein L15 mRNA, PCNA mRNA, ATRM gene exon 21, HR gene for hairless protein exon 2, N-terminal acetyltransferase complex and 1 subunit, HSM801431 homo sapiens mRNA, CDNA DKFZp434N2072,RPL26, and HR gene for hairless protein, regulator of G protein signaling.

Preferred methods include embodiments wherein an immune suppressive agent is administered prior to, concurrent with or subsequent to stem cell administration.

Preferred methods include embodiments wherein said immune suppressive agent is cyclophosphamide.

Preferred methods include embodiments wherein said immune suppressive agent is prednisone.

Preferred methods include embodiments wherein said immune suppressive agent is budesonide.

Preferred methods include embodiments wherein said immune suppressive agent is prednisolone.

Preferred methods include embodiments wherein said immune suppressive agent is tofacitinib.

Preferred methods include embodiments wherein said immune suppressive agent is cyclosporine.

Preferred methods include embodiments wherein said immune suppressive agent is tacrolimus.

Preferred methods include embodiments wherein said immune suppressive agent is everolimus.

Preferred methods include embodiments wherein said immune suppressive agent is azathioprine.

Preferred methods include embodiments wherein said immune suppressive agent is leflunomide.

Preferred methods include embodiments wherein said immune suppressive agent is Mycophenolate.

Preferred methods include embodiments wherein said immune suppressive agent is adalimumab.

Preferred methods include embodiments wherein said immune suppressive agent is anakinra.

Preferred methods include embodiments wherein said immune suppressive agent is certolizumab.

Preferred methods include embodiments wherein said immune suppressive agent is etanercept.

Preferred methods include embodiments wherein said immune suppressive agent is golimumab.

Preferred methods include embodiments wherein said immune suppressive agent is infliximab.

Preferred methods include embodiments wherein said immune suppressive agent is ixekizumab.

Preferred methods include embodiments wherein said immune suppressive agent is natalizumab.

Preferred methods include embodiments wherein said immune suppressive agent is rituximab.

Preferred methods include embodiments wherein said immune suppressive agent is secukinumab.

Preferred methods include embodiments wherein said immune suppressive agent is tocilizumab.

Preferred methods include embodiments wherein said immune suppressive agent is ustekinumab.

Preferred methods include embodiments wherein said immune suppressive agent is vedolizumab.

Preferred methods include embodiments wherein said immune suppressive agent is basiliximab.

Preferred methods include embodiments wherein said immune suppressive agent is daclizumab.

Preferred methods include embodiments wherein said at least one or more antioxidants and/or anti-inflammatory agents is administered before or after intraarticular laser treatment

### DETAILED DESCRIPTION OF THE INVENTION

The invention provides means of altering articular microenvironment in order to increase stem cell efficacy. Stem cell therapy for cartilage injury, in one embodiment of the invention, is autologous bone marrow derived stem cells. In one embodiment an antioxidant compound and/or anti-inflammatory compound is administered before stem cell therapy in order to augment possibility of stem cell success.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

As used herein, the word “a” or “plurality” before a noun represents one or more of the particular noun. For example, the phrase “a mammalian cell” represents “one or more mammalian cells.”

As used herein, the terms “subject” and “patient” are used interchangeably. A patient or a subject can be, for example and without limitation, a human subject, a racehorse or other mammals such as a companion animal (for example, a dog, a cat, etc.). A subject is any mammal that may benefit from the disclosed methods and compositions.

As used herein, the term “Progenitor” cell refers to a stem cell that is in a further stage of cell differentiation. Progenitor cells are unipotent or oligopotent and can get activated in response to injury and other cues, to initiate repair.

As used herein, the terms “chondroblast” refers to “Progenitor” cells that are partially or fully differentiated, and in essence, these two terms are used interchangeably. When positioned in the right milieu, chondroblasts will form chondrocytes. A chondroblast is a chondrocyte at an earlier stage of growth and development.

For the terms “for example” and “such as,” and grammatical equivalences thereof, the phrase “and without limitation” is understood to follow unless explicitly stated otherwise. As used herein, the term “about” is meant to account for variations due to experimental error. All measurements reported herein are understood to be modified by the term “about,” whether or not the term is explicitly used, unless explicitly stated otherwise. As used herein, the singular forms “a,” “an,” and “the” include plural referents unless the context clearly dictates otherwise.

The term “effective amount” or “a therapeutically effective amount” refers to an amount of an agent that provides a beneficial effect to a patient. The term “effective amount” or “a therapeutically effective amount” refers to an amount of an agent that provides the desired biological, therapeutic, and/or prophylactic result. That result can be reduction, amelioration, palliation, lessening, delaying, and/or alleviation of one or more of the signs, symptoms, or causes of a disease or disorder in a patient, or any other desired alteration of a biological system. A beneficial effect can take the form of an improvement over baseline, i.e., an improvement over a measurement or observation made prior to initiation of therapy according to the method. An effective amount or a therapeutically effective amount can be administered in one or more administrations.

The term “autologous” refers to the use of the stem cells, harvested from the same subject who receives it.

The term “allogeneic” refers to the use of the stem cells when the donor is different from the recipient.

The term “anterior cruciate ligament” (ACL) refers to the ligament that attaches the front of the bone of the lower leg, tibia, to the back of the bone of the thigh, femur, in each knee.

The term “posterior cruciate ligament” (PCL) also refers to the ligament that connects tibia with femur, but runs behind the ACL.

The term “medial collateral ligament” (MCL) refers to the ligament that attaches the medial lower tip of the femur to the medial upper tip of the tibia

The term “lateral collateral ligament” (LCL) refers to the ligament located on the outside of the knee joint, connecting the bottom of femur to the top of the smaller lower leg bone, fibula.

The term“meniscus” refers to a c-shaped cartilage pad located in the knee joint between femur and tibia.

Osteoarthritis (OA) is the most common form of arthritis and is developed when the protective hyaline cartilage on the ends of the bones wear down in time. Repetitive movements, heavy lifting, weakness of muscles associated with the joints and athletic injuries can also lead to cartilage breakdown and OA. Osteoarthritis causes pain, inflammation, and reduced motion in all joints, but mostly in the joints of the knees, hips, shoulders, hands and spine. An estimated 10%-15% of all adults aged over 60 will develop some degree of OA (over 30 million adults in the US), with prevalence being higher among women, and on the rise due to the ageing of the populations and obesity. OA is also a source of morbidity and economic loss in the racehorse and companion animal populations.

Aside from OA, a large population of younger subjects are afflicted with injuries to cartilage or ligaments of the joints. Common acute injuries of the joint involve damage to anterior and posterior cruciate ligament (ACL and PCL), medial and lateral collateral ligaments (MCL and LCL), Patellar cartilage and menisci.

Although pain, reduced mobility and other symptoms of damaged joints can be temporarily managed by routine modalities (pain killers, injection of steroids or hyaluronic acid in the joints, etc.), the underlying cause of the disease persists. Advanced cell therapy treatments have sought to replace the damaged cartilage and other components of the joints and restore the normal joint functions. Cells commonly used in regeneration include autologous mesenchyme stem cells derived from bone marrow, adipose tissue and full grown cartilage. Issues associated with the use of these sources include limited collection site and fully differentiated cells with low regenerative capacity. Also used for stem cell therapy are heterologous sources including umbilical, embryonic or placental tissues. These modalities involve complicated harvesting processes, possible immunological responses, and fewer than optimal number of compatible cells. Within the current invention, regeneration of cartilage is provided by administration of anti-oxidant and/or anti-inflammatory agents in order to increase receptivity of the articular microenvieronment for stem cell administration. “Cartilage,” or “cartilaginous tissue,” as used herein, encompasses articular cartilage, hyaline cartilage, neocartilage, devitalized cartilage, auricular cartilage, cartilage from an autogenous source, cartilage from an allogenic source, cartilage from a xenogeneic source, juvenile cartilage, tissue from the transient cartilaginous phase during bone formation and regeneration, or a combination thereof. The term “cartilaginous tissue” includes permanent as well as transient cartilage. For example, permanent cartilage includes or refers to articular cartilage, e.g., cartilage present at the interface between articulating bones such as knee, elbow, shoulder, spine, hip, finger, and/or toe bones. Transient cartilaginous tissue includes cartilage present in the growth plate of developing bone, e.g., cartilage that forms a template for bone in growing mammals such as humans. For example, a growth plate maintains a cartilaginous state up until the individual attains skeletal maturity, typically at the age of 16-25 years of age. Transient cartilaginous tissue also encompasses cartilage of regenerating bone, e.g., bone tissue that has been stressed, compromised, or injured, e.g., by a bone fracture, in an adult or juvenile individual. Bone regeneration in such circumstances, e.g., bone fracture healing, recapitulates bone development. For example, healing of a fractured bone includes a cartilage phase (cartilaginous tissue), which is then remodeled, resulting in healing and replacement of bone tissue at the site of the incident of bone stress, injury, or fracture.

In one embodiment reduction of inflammation in the intra-articular tissue is achieved by administration of various agents which includ redox-active compounds, iron-ligand inhibitors, flavonoids, corticosteroids, and nonsteroidal anti-inflammatory drugs. The live tissue solution also preferably includes compounds or molecules that modulate pathways associated with lipotoxicity, which could include fibrates and thiazolidinediones. Compounds or molecules that reduce reactive oxygen species, oxidation of lipids, and lipid peroxidation which include various antioxidants which could be used in combination are also preferably added to the solution. Examples include zinc, vitamin C (ascorbic acid), vitamin E (alpha-tocopherol), selenium, Trolox, ebselen, glutathione, carotenes, ubiquinol (Coenzyme Q), propyl gallate (PG), butylated hydroxytoluene (BHT), butylated hydroxyanisole (BHA), hydrogen sulfide, erythrobate, sodium tripolyphosphate, ethylenediaminetetraacetic acid, ethoxyquin, caseinates, pyruvate, natural herbs, honey, and similar compounds that inhibit oxidation. Other immune modulators that may be used together with antioxidants to modify the articular niche include: FAS ligand, IL-2R, IL-1 Ra, IL-2, IL-4, IL-8, IL-10, IL-20, IL-35, HLA-G, PD-L1, I-309, IDO, iNOS, CD200, Galectin 3, sCR1, arginase, PGE-2, aspirin, atorvastatin, fluvastatin, lovastatin, pravastatin, rosuvastatin, simvastatin, pitavastatin, n-acetylcysteine, rapamycin, IVIG, naltrexone, TGF-beta, VEGF, PDGF, CTLA-4, anti-CD45RB antibody, hydroxychloroquine, leflunomide, auranofin, dicyanogold, sulfasalazine, methotrexate, glucocorticoids, etanercept, adalimumab, abatacept, anakinra, certolizumab, Etanercept-szzs, golimumab, infliximab, rituximab, tocilizumab, cyclosporine, IFN-gamma, everolimus, rapamycin, or combinations thereof.

In other embodiments inhibitors of nitric oxide are utilized to modulate the microenvironment of the joint before, during and after stem cell administration. comprise nitric oxide synthase inhibitors and nitric oxide scavangers comprising; arginine derivatives, methylated arginines, substituted L-arginine, nitro-arginine, L-N.sup.G-nitroarginine, N.sup.G-mono-methyl-L-arginine (L-NMMA), N-nitro-L-arginine methyl ester (L-NAME), N-amino-L-arginine, N-methyl-L-arginine, N.sup.G-monomethyl-L-arginine (L-NMA), N.sup.G-nitro-L-arginine (L-NNA), aminoguanidine, 7-nitroindazole, S-ethylisothiourea, S-methylisothiourea, S-methylthiocitriulline, S-ethylthiocitrulline, N-ethylimino-L-ornithine, N-iminoethyl-L-lysine (L-NIL), flavoprotein binders. diphenyleneiodonium and related iodonium derivatives, omithine and omithine derivatives; tetracycline and derivaties thereof; L-canavanine; citrulline; redox dyes, methylene blue; calmodulin binders, trifluoropiperazine and calcinarin; heme binders; resveratrol; zinc compounds; tetrahydropterin analogs, aminoguanidine; and depleters of biopterin, methotrexate, N-acetylcysteine, nonsteroidal anti-inflammatory agents, sodium salicylate, and mixtures thereof. In some embodiments other pharmaceutically active ingredients are utilized to modulate the arthritic microenvironment including: pharmaceutically active agents selected from the group consisting of growth factors, differentiation factors, enzymes, receptor agonists or antagonists, antibodies, hormones, analgesics, local anesthetics, anti-inflammatory drugs, such as Indomethacin and tiaprofenic acid, TNF-.alpha. inhibitors, antibiotics, anti-microbial agents; antibiotics; antiproliferative, cytotoxic, and antitumor drugs including chemotherapeutic drugs; analgesic; antiangiogen; antibody; antivirals; cytokines; colony stimulating factors; proteins; chemoattractants; EDTA; histamine; antihistamine; erythropoietin; antifungals; antiparasitic agents; non-corticosteroid anti-inflammatory agents; anticoagulants; anesthetics including local anesthetics such as lidocaine and bupivicaine; analgesics; oncology agents; cardiovascular drugs; nutritional supplements; hormones; glycoproteins; fibronectin; peptides; interferons; cartilage inducing factors; protease inhibitors; vasoconstrictors, vasodilators, demineralized bone or bone morphogenetic proteins; hormones; lipids; carbohydrates; proteoglycans, versican, decorin, and biglycan; antiangiogenins; antigens; DBM; hyaluronic acid and salts and derivatives thereof; polysaccharides; cellulose compounds; antibodies; gene therapy reagents; genetically altered cells, stem cells; cell growth factors; type I and II collagen; collagen hydrolysate; elastin; sulfated glycosaminoglycan (sGAG), glucosamine sulfate; pH modifiers; methylsulfonylmethane (MSM); osteogenic compounds; osteoconductive compounds; plasminogen; nucleotides; oligonucleotides; polynucleotides; polymers; osteogenic protein 1 (OP-1 including recombinant OP-1); LMP-1 (Lim Mineralization Protein-1); cartilage including autologous cartilage; oxygen-containing components; enzymes such as, for example, peroxidase, which mediate the release of oxygen from such components; melatonin; vitamins; nutrients, and combinations thereof.

In some embodiments gene therapy is provided to induce an anti-oxidant environment prior to stem cell administration. In one embodiment gene therapy with superoxide dismutase is disclosed in order to modulate the microenvironment. The gene therapy can be administered in a higher dose to provide a systemic protective effect to stem cell compartments. One benefit of the systemic effect is that a dose of gene therapy can be administered to a patient and provide the desired effect at a variety of locations. This alleviates the need to locate all locations in need of treatment. The gene therapy can be administered via intravenous administration, intra-bone marrow administration, intra-arterial administration, intra-cardiac injection, intracerebral injection, intraspinal injection, intra-peritoneal injection, intra-muscular injection, subcutaneous injection, parenteral administration, intra-rectal administration, intra-tracheal injection, intra-nasal administration, intradermal injection, and the like. Administration of these compositions can be via any common route so long as the target tissue is available via that route.

The routes of administration will vary with the location and nature of damage to the stem cell compartment. One skilled in the art of preparing formulations can readily select the proper form and mode of administration depending upon the particular characteristics of the compound selected the disease state to be treated, the stage of the disease, and other relevant circumstances. The gene therapy can be administrated to the human individual or mammalian subject systemically, at the site of injury, at an adjacent site to the site of injury, and where following administrating the cells migrate to the site of injury. The details of the dosing schedule for the gene therapy are the amount necessary to provide the maximum selective protective effect upon exposure to ionizing radiation, which can be readily determined by one skilled in the art by the use of known techniques and by observing results obtained under analogous circumstances.

The vector for delivering superoxide dismutase may be any vector that may conveniently be subjected to recombinant DNA procedures, and the choice of vector will often depend on the host cell into which it is to be introduced. Thus, the vector may be an autonomously replicating vector, i.e. a vector that exists as an extrachromosomal entity, the replication of which is independent of chromosomal replication; examples of such a vector are a plasmid, phage, cosmid, mini-chromosome or virus. Alternatively, the vector may be one which, when introduced in a host cell, is integrated in the host cell genome and replicated together with the chromosome(s) into which it has been integrated. Additionally, the gene therapy can be administered in a higher dose to provide a systemic protective effect. The benefit of the systemic effect is that a dose of gene therapy can be administered to a patient and provide the desired effect at any necessary locations. This alleviates the need to locate all locations in need of treatment.

The gene therapy of the invention can be administered to the human or other animal after inflammation induced stem cell damage such as in osteoarthritis in an amount that is effective for diminishing damage to the respiratory, gastrointestinal and the hematopoietic systems after sublethal irradiation or for increasing the survival rate after lethal inflammation. The gene therapy may also be effective when administered prior to or during exposure to inflammation. Another dosing regimen would include multiple doses given both prior and/or following the exposure to inflammation. Those of skill in the art are well aware of how to apply adenoviral delivery to in vivo and ex vivo situations. For viral vectors, one generally will prepare a viral vector stock. Depending on the kind of virus and the titer attainable, one will deliver 1 to 10, 10 to 50, 100-1000, or up to 1.times.10.sup.4, 1.times.10.sup.5, 1.times.10.sup.6, 1.times.10.sup.7, 1.times.10.sup.8, 1.times.10.sup.9, 1.times.10.sup.10, 1.times.10.sup.11, or 1.times.10.sup.12 infectious particles to the patient in a pharmaceutically acceptable composition as discussed below. Various routes are contemplated for osteoarthritis. Where discrete locations or tissues may be identified, a variety of direct, local and regional approaches may be taken. For example, an organ may be directly injected with the adenovirus. The adenovirus can be delivered by a catheter having access to the tissue. One may utilize the local vasculature to introduce the vector into the tissue or organ by injecting a supporting vein or artery. A more distal blood supply route also may be utilized. It may also be beneficial to treat the surrounding tissue, not just the affected tissue. Where clinical applications are contemplated, it will be necessary to prepare pharmaceutical compositions in a form appropriate for the intended application. Generally, this will entail preparing compositions that are essentially free of pyrogens, as well as other impurities that could be harmful to humans or animals. Appropriate salts and buffers can be used to render delivery vectors stable and allow for uptake by target cells. Aqueous compositions of the gene therapy can include an effective amount of the vectors, dissolved or dispersed in a pharmaceutically acceptable carrier or aqueous medium. Such compositions also are referred to as inocula. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the gene therapy, its use in therapeutic compositions is contemplated. Supplementary active ingredients also can be incorporated into the compositions. An effective amount of the therapeutic agent is determined based on the intended goal, for example, lessening of cellular damage. The term “unit dose” refers to physically discrete units suitable for use in a subject, each unit containing a predetermined-quantity of the gene therapy composition calculated to produce the desired responses, discussed above, in association with its administration, i.e., the appropriate route and treatment regimen. The quantity to be administered, both according to number of treatments and unit dose, depends on the subject to be treated, the state of the subject, and the protection desired. Precise amounts of the therapeutic composition also depend on the judgment of the practitioner and are peculiar to each individual. The engineered viruses of the may be administered directly into animals, or alternatively, administered to cells that are subsequently administered to animals. The gene therapy may be administered parenterally or intraperitoneally. Solutions of the active compounds as free base or pharmacologically acceptable salts can be prepared in water suitably mixed with a surfactant, such as hydroxypropylcellulose. Dispersions also can be prepared in glycerol, liquid polyethylene glycols, and mixtures thereof and in oils. Under ordinary conditions of storage and use, these preparations contain a preservative to prevent the growth of microorganisms. The gene therapy compositions are advantageously administered in the form of injectable compositions either as liquid solutions or suspensions; solid forms suitable for solution in, or suspension in, liquid prior to injection may also be prepared. These preparations also may be emulsified. A typical composition for such purpose comprises a pharmaceutically acceptable carrier. For instance, the composition may contain 10 mg, 25 mg, and 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters such as ethyloleate. Aqueous carriers include water, alcoholic/aqueous solutions, saline solutions, and parenteral vehicles such as sodium chloride or Ringer's dextrose. Intravenous vehicles include fluid and nutrient replenishers. Preservatives include antimicrobial agents, anti-oxidants, chelating agents and inert gases. The pH and exact concentration of the various components the pharmaceutical composition are adjusted according to well known parameters. When the route is topical, the form may be a cream, ointment, or salve. In a further embodiment of the invention, an adenovirus or a nucleic acid encoding an adenovirus of the gene therapy may be delivered to cells using liposome or immunoliposome delivery. The adenovirus or nucleic acid encoding an adenovirus may be entrapped in a liposome or lipid formulation. Liposomes may be targeted to a cell by attaching antibodies to the liposome that bind specifically to a cell surface marker on the cell. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, Targeted Diagn Ther. 4:87-103 [1991]). Also contemplated is a nucleic acid construct complexed with Lipofectamine (Gibco BRL).

1. A method of enhancing stem cell activity in the treatment of cartilage degenerative conditions though administering at least one or more antioxidants and/or anti-inflammatory agents before, at the same time has, and subsequent to stem cell therapy.

2. The method of claim 1, wherein said cartilage degenerative condition is immunologically mediated.

3. The method of claim 1, wherein said cartilage degenerative condition is mediated by injury.

4. The method of claim 1, wherein said antioxidant is super oxide dismutase.

5. The method of claim 4, wherein the gene encoding manganese dependent superoxide dismutase is administered intra-articularly.

6. The method of claim 5, wherein said gene encoding manganese dependent superoxide dismutase is transfected into articular tissue by means of a viral vector.

7. The method of claim 5, wherein said gene encoding manganese dependent superoxide dismutase is transfected into articular tissue by means of a hydrodynamic transfection.

8. The method of claim 5, wherein said gene encoding manganese dependent superoxide dismutase is transfected into articular tissue by means of a mRNA liposomal delivery.

9. The method of claim 5, wherein said gene encoding manganese dependent superoxide dismutase is transfected into articular tissue by means of naked DNA administration.

10. The method of claim 1, wherein said antioxidant is selenium.

11. The method of claim 1, wherein said antioxidant is glutathione.

12. The method of claim 1, wherein said antioxidant is xenon gas.

13. The method of claim 1, wherein said anti-inflammatory agent is an NF-kappa B inhibitor.

14. The method of claim 13, wherein said NF-kappa B inhibitor is selected from the group consisting of: Perrilyl alcohol, Protein-bound polysaccharide from basidiomycetes, Rocaglamides (Aglaia derivatives), 15-deoxy-prostaglandin J(2), Lead, Anandamide, Artemisia vestita, Cobrotoxin, Dehydroascorbic acid (Vitamin C), Herbimycin A, Isorhapontigenin, Manumycin A, Pomegranate fruit extract, Tetrandine (plant alkaloid), Thienopyridine, Acetyl-boswellic acids, 1′-Acetoxychavicol acetate (Languas galanga), Apigenin (plant flavinoid), Cardamomin, Diosgenin, Furonaphthoquinone, Guggulsterone, Falcarindol, Honokiol, Hypoestoxide, Garcinone B, Kahweol, Kava (Piper methysticum) derivatives, mangostin (from Garcinia mangostana), N-acetylcysteine, Nitrosylcobalamin (vitamin B12 analog), Piceatannol, Plumbagin (5-hydroxy-2-methyl-1,4-naphthoquinone), Quercetin, Rosmarinic acid, Semecarpus anacardiu extract, Staurosporine, Sulforaphane and phenylisothiocyanate, Theaflavin (black tea component), Tilianin, Tocotrienol, Wedelolactone, Withanolides, Zerumbone, Silibinin, Betulinic acid, Ursolic acid, Monochloramine and glycine chloramine (NH2Cl), Anethole, Baoganning, Black raspberry extracts (cyanidin 3-O-glucoside, cyanidin 3-O-(2(G)-xylosylrutinoside), cyanidin 3-O-rutinoside), Buddlejasaponin IV, Cacospongionolide B, Calagualine, Carbon monoxide, Cardamonin, Cycloepoxydon; 1-hydroxy-2-hydroxymethyl-3-pent-1-enylbenzene, Decursin, Dexanabinol, Digitoxin, Diterpenes, Docosahexaenoic acid, Extensively oxidized low density lipoprotein (ox-LDL), 4-Hydroxynonenal (HNE), Flavopiridol, [6]-gingerol; casparol, Glossogyne tenuifolia, Phytic acid (inositol hexakisphosphate), Pomegranate fruit extract, Prostaglandin A1, 20(S)-Protopanaxatriol (ginsenoside metabolite), Rengyolone, Rottlerin, Saikosaponin-d, and Saline (low Na+ istonic).

15. The method of claim 1, wherein an inhibitor of inflammatory cytokines is administered intra-articularly prior to, concurrent with, or subsequent to stem cell administration.

16. The method of claim 15, wherein said inflammatory cytokines are selected from the group consisting of: inflammatory cytokines are cytokines capable of inducing expression of genes in endothelial cells selected from a group comprising of: IL-6, Myosin 1, IL-33, Hypoxia Inducible Factor-1, Guanylate Binding Protein Isoform I, Aminolevulinate delta synthase 2, AMP deaminase, IL-17, DNAJ-like 2 protein, Cathepsin L, Transcription factor-20, M31724, pyenylalkylamine binding protein; HEC, GA17, arylsulfatase D gene, arylaulfatase E gene, cyclin protein gene, pro-platelet basic protein gene, PDGFRA, human STS WI-12000, mannosidase, beta A, lysosomal MANBA gene, UBE2D3 gene, Human DNA for Ig gamma heavy-chain, STRL22, BHMT, homo sapiens Down syndrome critical region, FI5613 containing ZNF gene family member, IL8, ELFR, homo sapiens mRNA for dual specificity phosphatase MKP-5, homo sapiens regulator of G protein signaling 10 mRNA complete, Homo sapiens Wnt-13 Mma, homo sapiens N-terminal acetyltransferase complex ardl subunit, ribosomal protein L15 mRNA, PCNA mRNA, ATRM gene exon 21, HR gene for hairless protein exon 2, N-terminal acetyltransferase complex and 1 subunit, HSM801431 homo sapiens mRNA, CDNA DKFZp434N2072,RPL26, and HR gene for hairless protein, and regulator of G protein signaling.

17. The method of claim 1, wherein an immune suppressive agent is administered prior to, concurrent with or subsequent to stem cell administration.

18. The method of claim 17, wherein said immune suppressive agent is tacrolimus.

19. The method of claim 17, wherein said immune suppressive agent is everolimus.

20. The method of clam 1, wherein said at least one or more antioxidants and/or anti-inflammatory agents is administered before or after intraarticular laser treatment.
